(19) United States
(12) Patent Application Publication
(10) Pub. No.: US 2007/0255425 A1
(43) Pub. Date: Nov. 1, 2007

(54) Connection Part for Prostheses

(75) Inventor: Michael Weihermuller, Bayreuth (DE)

Correspondence Address: Thomas R. Vigil;Pyle & Piontek, 221 N. LaSalle Street, Room 2036, Chicago, IL, 60601, US

(73) Assignee: MEDI BAYREUTH WEIHERMULLER & VOIGTMANN GMBH & CO. KG, BAYREUTH (DE)

(21) Appl. No.: 11/597,024

(22) PCT Filed: May 17, 2005
(86) PCT No.: PCT/DE05/00893

§ 371 Date: Jul. 6, 2007

(30) Foreign Application Priority Data
May 19, 2004 (DE) 20 2004 008 159.8

(51) Int. Cl. A61F 2/60

(52) U.S. Cl. 623/27

(57) ABSTRACT

The invention relates to a connection part for prostheses, in particular a locking member (6) comprises a bore with a spring-loaded catch for a grooved or ratcheted contour of the pin (4) and a release device for the locking member that can be actuated in opposition to the force of the spring. The locking member (6) also comprises an integrated clamping mechanism for a cord (21) of a prosthetic liner, without a pin, said cord being pulled to introduce the liner into the prosthetic shaft.

The present invention relates to a connection part for prostheses having the features of the preamble of claim 1.

Such a connection part for a leg prosthesis is known from DE 197 18 580 A1 for example. Here, a prosthetic liner has a pin for insertion into a connection part connected to a shank adapter which in turn is connected to the prosthesis tube through a clamping element. The connection part has an unlockable, spring-loaded toothed latch for a toothed portion of said pin. The documents GB 2 338 899 A or DE 93 21 342 U1 have a very similar structure. In all the cases, there are provided unlocking rods that are actuatable from the outside and by means of which the locking element can be moved out of locking engagement with the pin. The document DE 100 26 399 A1 shows a connection part that may be optionally equipped as described herein above but that also offers the possibility of using a pull-in cord for the liner, a catch for the pull-in cord being located on the prosthesis shank. Switching between the alternatives requires quite a complex modification.

It is the object of the present invention to provide a connection part for prostheses, more specifically of the type last mentioned, which is configured to be variable and obviates the need for modifying the structure when switching between a solution using an insertion pin and one using a pull-in cord.

This object is solved with the features of the characterizing portion of claim 1. Improvements and advantageous implementations of the invention are comprised herein after.

In accordance with the invention, there is provided a connection part for prostheses, more specifically a locking member for a pin of a prosthetic liner, said locking member comprising a hole with a spring-loaded latch for a groove or ratchet shaped contour of said pin, as well as a release device for releasing the locking member, said release device being actuatable against the force of said spring, characterized in that the locking member moreover comprises a clamping mechanism integrated therein for a cord of a prosthetic liner that has no pin and is insertable into the prosthesis shank by means of said cord.

According to a preferred embodiment of the invention, the release device consists of an unlocking rod that has at its outer end an unlocking head guided toward the outside and is guided so as to glide in a channel of the locking member, said locking rod engaging at its inner end with a fixation forming the latch, said fixation being displaceably disposed in a recess of the locking member and said fixation being biased by the force of at least one spring toward a locking position and toward the inner end of the unlocking rod, a displacement of the unlocking rod into an unlocking position toward the inside of the locking member causing the fixation to be carried along with said rod and the pin to come free. The unlocking rod is guided in the channel so as to be secured from being lost.

In accordance with the invention, the locking member has a hole, recess or the like for guiding the cord through the hole out of the locking member. According to the preferred implementation of the invention, the unlocking rod is connected in a central region with at least one cord clamping pawl for the cord, said cord clamping pawl being pivotally or displaceably disposed on the unlocking rod, said locking member having a recess for a work region of the cord clamping pawl, said cord being guided through the recess and a wall, a projection of the recess or a part inserted therein serving as a clamping limit stop for the cord clamping pawl and stopping the cord in a final position of the work region of the cord clamping pawl, the remaining work region of the cord clamping pawl allowing the cord to come free and said cord clamping pawl only clamping the cord in the pulling direction, which is counter to the direction of pulling in the liner. The recess for the work region of the cord clamping pawl is disposed between two cord guides or a channel or little tube or the like serves for guiding the cord. According to an advantageous embodiment of the invention, the cord clamping pawl is biased by the force of a spring in the direction of the clamping limit stop. A release limit stop for the cord clamping pawl is preferably disposed in the recess, a displacement of the unlocking rod into the unlocking position causing the cord clamping pawl to be pushed against the release limit stop and to rotate about its articulation at the locking position into the remaining work region and the cord to come free.

According to an advantageous embodiment of the invention, there is provided a valve for evacuating air from the connection part and/or for allowing air to penetrate thereinto; said valve may be configured to be manually actuatable.

The connection part can be screwed or the like onto the prosthesis shank; it may also be cast in the distal end thereof.

The invention will be described in closer detail hereinafter by way of example with reference to drawings. In said drawings:

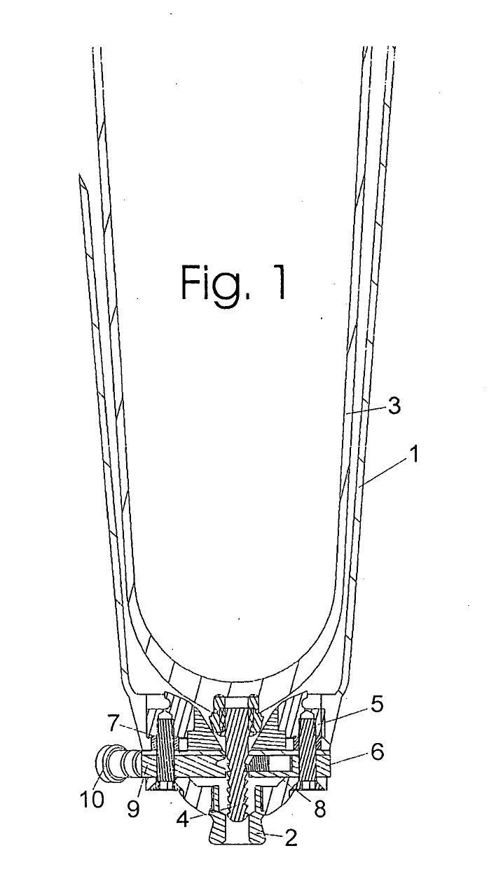

FIG. 1 is a sectional view of a prosthesis shank with connection part and inserted liner;

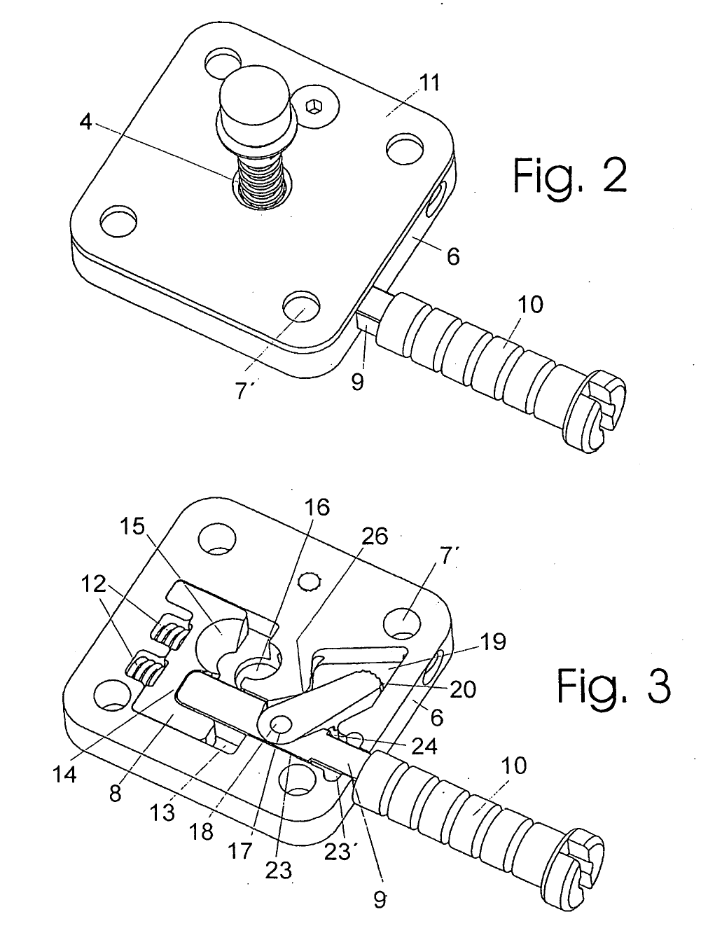

FIG. 2 shows a connection part with a locking member;

FIG. 3 shows the locking member of FIG. 2 in an opened position;

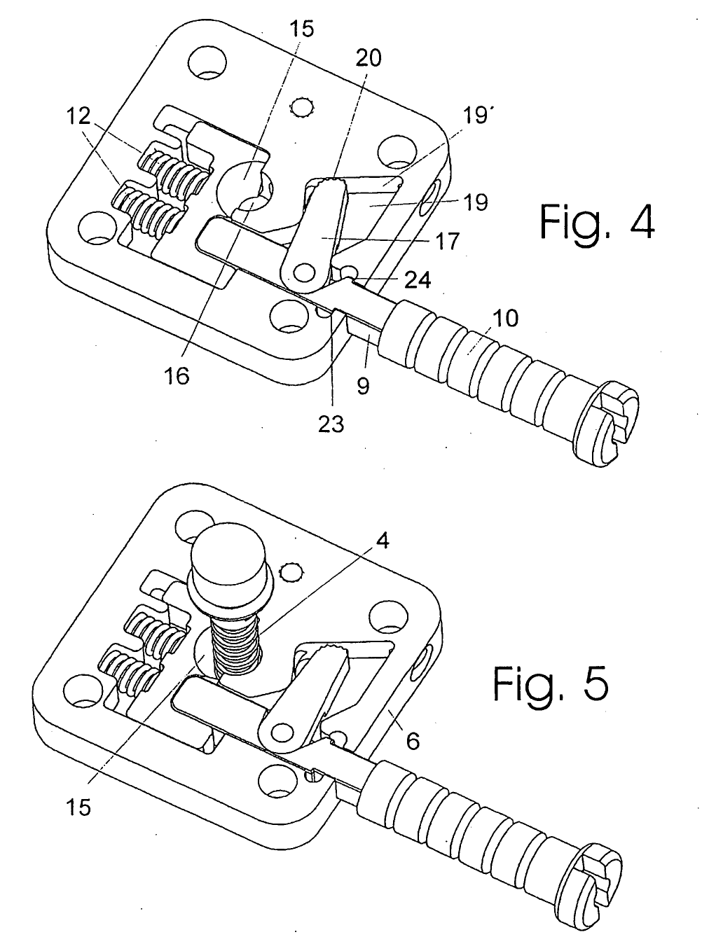

FIG. 4 shows the locking member of FIGS. 2 and 3 in the closed position;

FIG. 5 shows the position of FIG. 4 with the pin of a liner inserted;

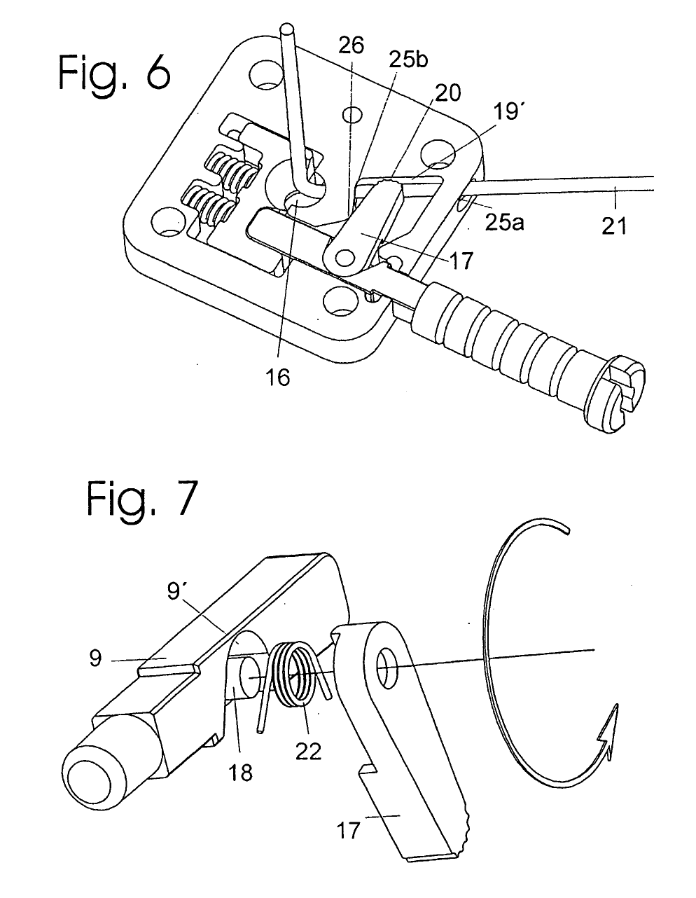

FIG. 6 shows the locking member of FIGS. 2 through 5 with the pull-in cord of a liner being clamped;

FIG. 7 is a detail view of the cord clamping pawl of FIG. 6.

FIG. 1 shows a prosthesis shank 1 with a shank adapter 2 for connection to an artificial knee joint (not shown). A liner 3 receiving the leg stump (not shown) of the user is inserted into the prosthesis shank 1. The liner 3 is an elastic cover made from silicone for example and has, at its distal end, a notched, grooved or toothed pin 4 by means of which it is retained in a locking member 6. As illustrated in FIG. 2, the locking member 6 is covered at the top by a plate 11 and has through holes 7′ for screws 7 by means of which the shank adapter 2 fitting against the locking member 6 is screwed from the bottom onto the distal end of the shank 1.

The FIGS. 3 through 5 show structure and function of the locking member 6 during insertion of the pin 4 of the liner 3. For fixing and retaining the pin 4, it has a latch that is formed by the insertion hole 16 for the pin 4 and by a fixating element 14 that is slidably carried in the locking member. The locking element 6 has a recess 13 in which the fixating element 14 is carried. The recess 13 has an approximately semi-circular bight portion which bounds the insertion hole 16 so as to allow the pin 4 to move freely inward and outward along the wall thereof. Opposite said bight portion, there is disposed the fixating element 14, which is horizontally displaceable within the recess 13. It comprises an also approximately semi-circular upward flaring bight portion for ratchet-like engagement with the corrugated structure of the pin 4. On the side confronting the bight portion 15, the fixating element 14 abuts the wall of the recess 13 by means of two springs 12, thus being biased toward the closed position shown in FIG. 2. In this closed position, the corrugated pin 4 can be inserted in the thus formed latch since it is capable of displacing the fixating element 14 on its funnel-shaped bight portion 15 against the force of the springs 12 but can no longer be pulled out since the lower rim of the funnel-shaped bight portion 15 is urged by the springs 12 to lie behind the respective grooves of the pin 4. An unlocking rod 9 capable of bringing the locking member 6 into the opened position shown in FIG. 3 is therefore provided for releasing the pin. The unlocking rod 9 is horizontally slidably guided in a channel 23 that leads outward from the recess 13. The inner end of the unlocking rod 9 fits beside the latch formed by the funnel-shaped bight portion 15 and the bight portion for the insertion hole 16 against the fixating element 14 and is capable of urging the latter backward against the force of the springs 12 when a user pushes an unlocking button 10 that is disposed outside the locking member 6 as a continuation of the unlocking rod 9. The unlocking rod 9 has shoulders 24 by means of which it is secured from falling out on limit stops 23′ of the channel 23.

The FIGS. 3 and 6 show how, instead of a pin 4, a pull-in cord 21 of a liner can be clamped in the locking member 6. For this purpose, the locking member 6 has a second recess 19 that is disposed in front of the recess 13 when viewed from the unlocking button 10 and forms a work region for a cord clamping pawl 17. The cord clamping pawl 17 is hinged to the unlocking rod 9 for rotation about a pivot 18. In the closed position of the locking member 6 shown in the FIGS. 4 and 6, the cord clamping pawl 17 fits against a wall 19′ of the recess 19 at its end located opposite its articulation point 18 at the locking rod 9. For this purpose, the cord clamping pawl 17 is biased toward this position by a spiral spring 22 that is disposed about the pivot 18 of the unlocking rod 9 and that abuts in a recess 9′ of the unlocking rod (FIG. 7). FIG. 6 shows how the cord 21 is guided in the locking member 6. At one end, it is fastened on the liner (not shown) and enters the insertion hole 16 and from there passes through a hole 25b in the second recess 19 and along the wall 19′ thereof through a second hole 25a to exit the locking member 6. At the wall 19′, it is clamped by the cord clamping pawl 17 so that it may be pulled out since the cord clamping pawl gives in this direction but cannot be pulled back since this would cause the cord clamping pawl 17 to be pulled onto the wall 19′ where it would jam. For this purpose, the front end of the cord clamping pawl 17 is equipped with a corrugated structure 20. The release of the cord also occurs by pushing the unlocking button and, as a result thereof, by displacing the unlocking rod 9 inward, as already described with regard to FIG. 3. A releasing limit stop 26 that is configured in the shape of a projection in the second recess 19 and that stops travel of the cord clamping pawl 17 in a central region when the unlocking rod is moving inward thereby comes into function. If the unlocking rod is further pushed inward, the cord clamping pawl 17 is caused to rotate out of its clamping position the end point of which is shown in FIG. 3. The cord has now come free and can be pulled through the insertion hole 16 upward and out of the locking member 6.

With the double acting mechanism for both clamping and releasing a pin and a cord with in parts the same components, the present invention provides a connection part for prostheses that permits to switch between the two solutions at little expense.

1. A connection part for prostheses, more specifically a locking member (6) for a pin (4) of a connection part on a prosthetic liner, said locking member (6) comprising a hole with spring-loaded latch for a groove or ratchet shaped contour of said pin (4), as well as a release device for releasing the locking member, said release device being actuatable against the force of said spring, characterized in that said locking member (6) moreover comprises a clamping mechanism integrated therein for a cord (21) of a prosthetic liner that has no pin and is insertable into the prosthesis shank by means of said cord.

2. The connection part as set forth in claim 1, characterized in that the release device consists of an unlocking rod (9) that has at its outer end an unlocking head (10) guided toward the outside and is guided so as to glide in a channel (23) of the locking member (6), that said locking rod (9) engages at its inner end with a fixation (14) forming the latch, that said fixation (14) is displaceably disposed in a recess (13) of the locking member and that said fixation (14) is biased by the force of at least one spring (12) toward a locking position and toward the inner end of the unlocking rod (9), a displacement of the unlocking rod into an unlocking position toward the inside of the locking member (6) causing the fixation (14) to be carried along with said rod and the pin (4) to come free.

3. The connection part as set forth in claim 1, characterized in that the unlocking rod (9) is guided in the channel (23) so as to be secured from being lost.

4. The connection part as set forth in claim 1, characterized in that the locking member (6) has a hole, recess or the like for guiding the cord (21) through the hole out of the locking member.

5. The connection part as set forth in claim 4, characterized in that the unlocking rod (9) is connected in a central region with at least one cord clamping pawl (17) for the cord (21), that said cord clamping pawl (17) is pivotally or displaceably disposed on said locking rod (9), that said locking member (6) has a recess (19) for a work region of the cord clamping pawl (17), that said cord (21) is guided through the recess (19) and that a wall (19′), a projection of the recess or a part inserted therein serves as a clamping limit stop for the cord clamping pawl (17) and stops the cord (21) in a final position of the work region of the cord clamping pawl (17), the remaining work region of the cord clamping pawl allowing the cord to come free and said cord clamping pawl (17) only clamping the cord (21) in the pulling direction, which is counter to the direction of pulling in the liner.

6. The connection part as set forth in claim 5, characterized in that the recess (19) for the work region of the cord clamping pawl is disposed between two cord guides or that a channel or a little tube or the like serves for guiding the cord.

7. The connection part as set forth in claim 5, characterized in that the cord clamping pawl (17) is biased by the force of a spring (22) in the direction of the clamping stop limit.

8. The connection part as set forth in claim 6, characterized in that a release limit stop (26) for the cord clamping pawl is disposed in the recess (19), and that a displacement of the unlocking rod (9) into the unlocking position causes the cord clamping pawl (17) to be pushed against the release limit stop (26) and to rotate about its articulation at the locking position into the remaining work region and the cord (21) to come free.

9. The connection part as set forth in claim 1, characterized in that a valve for evacuating the air from the connection part and/or for allowing air to penetrate thereinto is provided on the connection part.

10. The connection part as set forth in claim 9, characterized in that said valve is manually actuatable.
